(19) United States
(12) Patent Application Publication
(10) Pub. No.: US 2009/0118716 A1
(43) Pub. Date: May 7, 2009

(54) SYSTEM AND METHOD FOR SCANNING A PULSED LASER BEAM

(75) Inventor: Michael Brownell, San Clemente, CA (US)

Correspondence Address: ADVANCED MEDICAL OPTICS, INC., 1700 E. ST. ANDREW PLACE, SANTA ANA, CA, 92705, US

(73) Assignee: IntraLase, Inc., Santa Ana, CA (US)

(21) Appl. No.: 11/936,298

(22) Filed: Nov. 7, 2007

(51) Int. Cl. A61F 9/01; G02B 5/08; H01S 3/10

(52) U.S. Cl. 606/4; 359/838; 372/24; 372/25

(57) ABSTRACT

System and method of photoaltering a region of a material using a pulsed laser beam. The method includes randomly scanning the pulsed laser beam in the region, and creating a separation between a first layer of the material and a second layer of the material at the region. The system includes a laser producing a pulsed laser beam, a controller transmitting a signal, and a scanner coupled to the controller. The scanner randomly scans the pulsed laser beam in the region in response to the signal.

### BACKGROUND OF THE INVENTION

1. Field of the Invention

The field of the present invention is generally related to photoaltering materials and more particularly, to systems and methods for scanning pulsed laser beams.

2. Background

Pulsed laser beams include bursts or pulses of light, as implied by name, and have been used for photoalteration of materials, both inorganic and organic alike. Typically, a pulsed laser beam is focused onto a desired area of the material to photoalter the material in this area and, in some instances, the associated peripheral area. Examples of photoalteration of the material include, but are not necessarily limited to, chemical and physical alterations, chemical and physical breakdown, disintegration, ablation, vaporization, or the like.

One example of photoalteration using pulsed laser beams is the photodisruption (e.g., via laser induced optical breakdown) of a material. Localized photodisruptions can be placed at or below the surface of the material to produce high-precision material processing. For example, a micro-optics scanning system may be used to scan the pulsed laser beams to produce an incision in the material and create a flap therefrom. The term “scan” or “scanning” refers to the movement of the focal point of the pulsed laser beam along a desired path or in a desired pattern. To create a flap of the material, the pulsed laser beam is typically scanned along a pre-determined region (e.g., within the material) in either a spiral pattern or a raster pattern. In general, these patterns are mechanically simple to implement (e.g., continuous) and control for a given scan rate and desired focal point separation of the pulsed laser beam.

Limitations of the scanning system, such as due to mechanical restrictions, may preclude placing high density, low energy pulses into a desired region. For example, for opthalmic applications, the scanning system may be precluded from placing high density, low energy pulses on or in a desired region of the cornea. In some cases, these limitations may also limit dissection quality improvements and power (e.g., average power) reductions in corneal procedures. Advanced flap geometries (e.g., more complicated flap shapes) and procedures associated with these geometries compound such limitations.

Accordingly, it is desirable to provide a system and method for photoaltering a material that improves dissection quality and reduces the average power associated with such dissections. It is also desirable to provide a system and method for photoaltering a region of material having a variety of geometries. It is also desirable to provide a system and method for creating a flap of a material with a pulsed laser beam that improves dissection quality and power reduction associated with flap creation. Additionally, other desirable features and characteristics of the present invention will become apparent from the subsequent detailed description and the appended claims, taken in conjunction with the accompanying drawings and the foregoing technical field and background.

### SUMMARY OF THE INVENTION

The present invention is directed towards photoaltering a material using a pulsed laser beam. In one embodiment, a method of photoaltering a region of the material using a pulsed laser beam is provided. The method includes randomly scanning the pulsed laser beam in the region, and creating a separation between a first layer of the material and a second layer of the material at the region.

In another embodiment, a system for photoaltering a region of the material is provided. The system includes a laser configured to produce a pulsed laser beam, a controller configured to transmit a signal, and a scanner coupled to the controller. The scanner is operable to randomly scan the pulsed laser beam in the region in response to the signal.

In another embodiment, a method of forming a corneal flap of an eye is provided. The method includes randomly scanning a corneal region of the eye with a pulsed laser beam, the corneal region having a periphery, and incising at least a portion of the periphery with the pulsed laser beam.

### BRIEF DESCRIPTION OF THE DRAWINGS

In the drawings, wherein like reference numerals refer to similar components:

FIG. 1 is a block diagram of a laser scanner system in accordance with one embodiment of the present invention;

FIG. 2 is an elevational view of a laser scanner system in accordance with one embodiment;

FIG. 3 is a perspective view of a cornea and a stromal bed in the cornea; and

FIG. 4 is a flow diagram of a method for photoaltering a material in accordance with one embodiment.

### DETAILED DESCRIPTION

The present invention provides systems and methods for scanning a pulsed laser beam that places high density, low energy pulses in a desired area or region. Photoalteration of a material may be accomplished using a pulsed laser beam that is directed (e.g., via a scanner) at a desired region of the material. For example, a pulsed laser beam may be controlled to scan the desired region and to create a separation of the material (e.g., which may be used to produce a flap of the material). To impart at least a portion of this control, software, firmware, or the like, can be used to command the actions and placement of the scanner via a motion control system, such as a closed-loop proportional integral derivative (PID) control system. In one embodiment, the pulsed laser beam is randomly scanned with a low pulse energy and low average pulse energy in the desired region. The term “random” or “randomly” as used herein with scanning or patterns is defined herein to mean a substantially undirected scan spot placement, such as a spray or the like. The result is a pattern in the desired region having a relatively high scan spot density and locally randomized scan spot distribution.

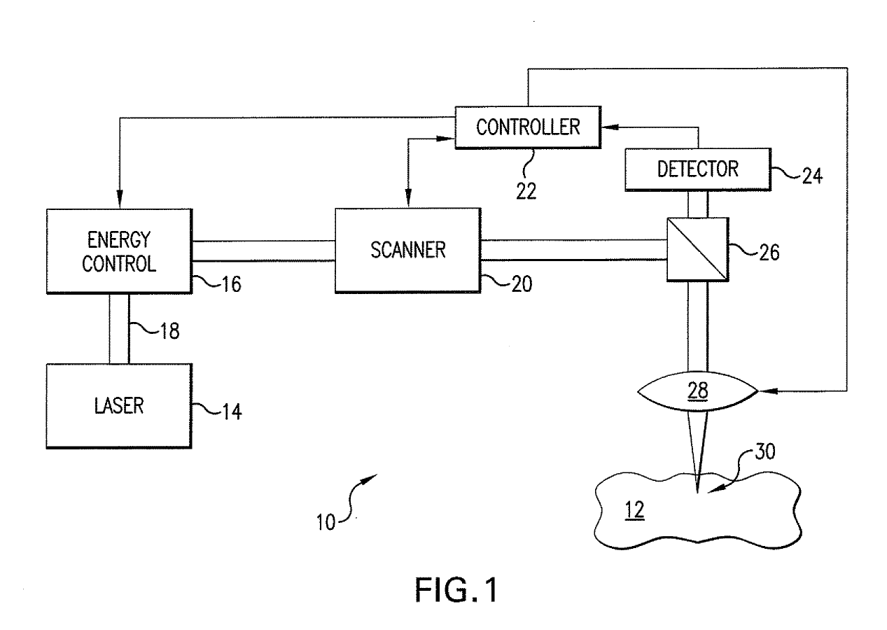

Referring to the drawings, a system 10 for photoaltering a material 12 is shown in FIG. 1. The system 10 includes, but is not necessarily limited to, a laser 14 capable of generating a pulsed laser beam 18, an energy control module 16 for varying the pulse energy of the pulsed laser beam 18, a scanner 20, a controller 22, and focusing optics 28 for directing the pulsed laser beam 18 from the laser 14 on the surface of or within the material 12 (e.g., sub-surface). The controller 22 (e.g., a processor operating suitable control software) communicates with the scanner 20 and/or focusing optics 28 to direct a focal point 30 of the pulsed laser beam onto or into the material 12. In this embodiment, the system 10 further includes a beam splitter 26 and a detector 24 coupled to the controller 22 to provide a feedback control mechanism for the pulsed laser beam 18. The beam splitter 26 and detector 24 may be omitted in other embodiments, for example, with different control mechanisms.

Movement of the focal point of the pulsed laser beam 18 is accomplished via the scanner 20 in response to the controller 22. In one embodiment, the scanner 20 randomly scans the pulsed laser beam 18 in at least a portion of the desired region. By randomly scanning the pulsed laser beam 18, a group of scan spots are produced in the region, and this group is characterized by a high scan spot density.

In addition to random scanning, the scanner 20 may selectively move the focal point of the pulsed laser beam 18 to produce a structured scan pattern (e.g., a raster pattern, a spiral pattern, or the like) via the controller 22. Operating the scanner 20 to scan this structure pattern is particularly useful for controlling the spacing between scan spots of the pattern. The step rate at which the focal point is moved is referred to herein as the scan rate. For example, the scanner 20 can operate at scan rates between about 10 kHz and about 400 kHz, or at any other desired scan rate. In one embodiment, the scanner 20 generally moves the focal point of the pulsed laser beam 18 through the desired scan pattern at a substantially constant scan rate while maintaining a substantially constant separation between adjacent focal points of the pulsed laser beam 18. Further details of laser scanners are known in the art, such as described, for example, in U.S. Pat. No. 5,549,632, the entire disclosure of which is incorporated herein by reference.

To provide the pulsed laser beam, a chirped pulse laser amplification system, such as described in U.S. Pat. No. RE37,585, may be used for photoalteration. U.S. Pat. Publication No. 2004/0243111 also describes other methods of photoalteration. Other devices or systems may be used to generate pulsed laser beams. For example, non-ultraviolet (UV), ultrashort pulsed laser technology can produce pulsed laser beams having pulse durations measured in femtoseconds. Some of the non-UV, ultrashort pulsed laser technology may be used in ophthalmic applications. For example, U.S. Pat. No. 5,993,438 discloses a device for performing ophthalmic surgical procedures to effect high-accuracy corrections of optical aberrations. U.S. Pat. No. 5,993,438 discloses an intrastromal photodisruption technique for reshaping the cornea using a non-UV, ultrashort (e.g., femtosecond pulse duration), pulsed laser beam that propagates through corneal tissue and is focused at a point below the surface of the cornea to photodisrupt stromal tissue at the focal point.

Although the system 10 may be used to photoalter a variety of materials (e.g., organic, inorganic, or a combination thereof), the system 10 is suitable for ophthalmic applications in one embodiment. In this case, the focusing optics 28 direct the pulsed laser beam 18 toward an eye (e.g., onto a cornea) for plasma mediated (e.g., non-UV) photoablation of superficial tissue, or into the stroma for intrastromal photodisruption of tissue. In this embodiment, the system 10 may also include an applanation lens (not shown) to flatten the cornea prior to scanning the pulsed laser beam 18 toward the eye. A curved, or non-planar, lens may substitute this applanation lens to contact the cornea in other embodiments.

The system 10 is capable of generating the pulsed laser beam 18 with physical characteristics similar to those of the laser beams generated by a laser system disclosed in U.S. Pat. No. 4,764,930, U.S. Pat. No. 5,993,438, or the like. For example, the system 10 can produce a non-UV, ultrashort pulsed laser beam for use as an incising laser beam. This pulsed laser beam preferably has laser pulses with durations as long as a few nanoseconds or as short as a few femtoseconds. For intrastromal photodisruption of the tissue, the pulsed laser beam 18 has a wavelength that permits the pulsed laser beam 18 to pass through the cornea without absorption by the corneal tissue. The wavelength of the pulsed laser beam 18 is generally in the range of about 3 μm to about 1.9 nm, preferably between about 400 nm to about 3000 nm, and the irradiance of the pulsed laser beam 18 for accomplishing photodisruption of stromal tissues at the focal point is greater than the threshold for optical breakdown of the tissue. Although a non-UV, ultrashort pulsed laser beam is described in this embodiment, the pulsed laser beam 18 may have other pulse durations and different wavelengths in other embodiments.

In ophthalmic applications, the scanner 20 may utilize a pair of scanning mirrors or other optics (not shown) to angularly deflect and scan the pulsed laser beam 18. For example, scanning mirrors driven by galvanometers may be employed where each of the mirrors scans the pulsed laser beam 18 along one of two orthogonal axes. A focusing objective (not shown), whether one lens or several lenses, images the pulsed laser beam onto a focal plane of the system 10. The focal point of the pulsed laser beam 18 may thus be scanned in two dimensions (e.g., the x-axis and the y-axis) within the focal plane of the system 10. Scanning along the third dimension, i.e., moving the focal plane along an optical axis (e.g., the z-axis), may be achieved by moving the focusing objective, or one or more lenses within the focusing objective, along the optical axis.

When preparing a cornea for flap separation, for example, a circular area may be scanned using a scan pattern driven by the scanning mirrors. The pulsed laser beam 18 photoalters the stromal tissue as the focal point of the pulsed laser beam 18 is scanned in a corneal bed. Using structured patterns, the distribution of scan spots is determined by the pulse frequency, the scan rate, and the amount of scan line separation. Generally, higher scan rates, enable shorter procedure times by increasing the rate at which corneal tissue can be photoaltered. For example, the scan rates may be selected from a range between about 30 MHz and about 1 GHz with a pulse width in a range between about 300 picoseconds and about 10 femtoseconds, although other scan rates and pulse widths may be used.

The system 10 may additionally acquire detailed information about optical aberrations to be corrected, at least in part, using the system 10. Examples of such detailed information include, but are not necessarily limited to, the extent of the desired correction, and the location in the cornea of the eye associated with the correction (e.g., where the correction can be made most effectively). The refractive power of the cornea may be used to indicate corrections. Wavefront analysis techniques, made possible by devices such as a Hartmann-Shack type sensor (not shown), can be used to generate maps of corneal refractive power. Other wavefront analysis techniques and sensors may also be used. The maps of corneal refractive power, or similar refractive power information provided by other means, such as corneal topographs or the like, can then be used to identify and locate the optical aberrations of the cornea that require correction.

In general, when the laser 14 is activated, the focal spot 30 of the pulsed laser beam 18 is selectively directed (e.g., via the scanner 20) along a beam path to photoalter stromal tissue. For example, the focal spot 30 of the pulsed laser beam 18 is moved along a predetermined length of the beam path in one reference area. The pulsed laser beam 18 is then redirected through another reference area, and the process of photoalteration is repeated. The sequence for directing the pulsed laser beam 18 through individually selected reference areas can be varied, and the extent of stromal tissue photoalteration while the incising laser beam is so directed, can be varied. Specifically, as indicated above, the amount of photoalteration can be based on the refractive power map. On the other hand, the sequence of reference areas that is followed during a customized procedure will depend on the particular objectives of the procedure.

The scanner 20 may also scan a predetermined pattern using one or more scan patterns to one or more combinations of these reference areas or scan the pulsed laser beam as a single focal point (e.g., to produce a sidecut). One example of an ophthalmic scanning application is a laser assisted in-situ keratomilieusis (LASIK) type procedure where a flap is cut from the cornea to establish extracorporeal access to the tissue that is to be photoaltered. The flap may be created using random scanning or one or more scan patterns of pulsed laser beams. To create the corneal flap, a sidecut is created around a desired perimeter of the flap such that the ends of the sidecut terminate, without intersection, to leave an uncut segment. This uncut segment serves as a hinge for the flap. The flap is separated from the underlying stromal tissue by scanning the laser focal point across a resection bed, the perimeter of which is approximately defined by and slightly greater than the sidecut. Once this access has been achieved, photoalteration is completed, and the residual fragments of the photoaltered tissue are removed from the cornea. In another embodiment, intrastromal tissue may be photoaltered by the system 10 so as to create an isolated lenticle of intrastromal tissue. The lenticle of tissue can then be removed from the cornea.

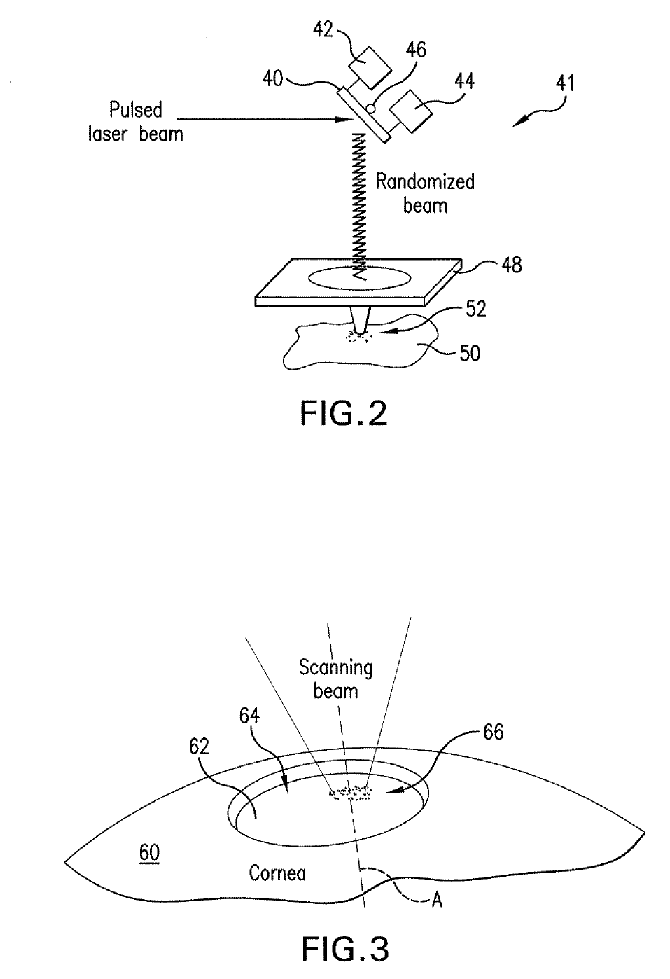

FIG. 2 is an elevational view of a laser scanner system 41, in accordance with one embodiment illustrating randomized scanning. In this embodiment, the system 41 includes a mirror 40, a piezo element 42 coupled to the mirror 40, and a biasing device 44 (e.g., a spring) coupled to the mirror 40 that operates in conjunction with the piezo element 42 to displace the mirror 40 about a pivot 46. The piezo element 42 and biasing device 44 are preferably coupled to the mirror 40 to displace the mirror 40 in at least one of two orthogonal axes or directions. For example, the piezo element 42 may be coupled to one end of the mirror 40 with the biasing device 44 coupled to an opposing end of the mirror 40 such that the mirror 40 displaces about the pivot 46 between these two ends. Although the piezo element 42 and biasing device 44 are used to illustrate randomized scanning, other mechanisms may be used that impart a substantially random yet controlled displacement of the mirror (e.g., randomized scan spots within a localized region).

Referring to FIGS. 1 and 2, the mirror 40, spring 44, pivot 46, and piezo element 42 may be incorporated as components of the scanner 20 in one embodiment. A pulsed laser beam, such as provided by the laser 14, is directed at the mirror 40. When random scanning is desired, the piezo element 42 is activated (e.g., via a signal received from the controller 22) to displace the mirror 40 in a substantially random manner, and the mirror 40 reflects the pulsed laser beam to produce a randomized beam that is directed through focusing optics 48, such as the focusing optics 28 shown in FIG. 1, to a desired region 52 of a material 50. As a result, the displacement of the mirror 40 randomizes the beam movement along at least one of two dimensions (e.g., along the x-axis, the y-axis, or both of the x- and y-axes) of the focal plane of the focusing optics 48, and a random scan spot pattern is produced in the region 52.

The randomized scanning may be used alone (e.g., to photoalter the entire region 52 of the material 50) or in combination with one or more scan patterns (e.g., one or more structured scan patterns such as a raster or spiral pattern) to scan the entire region 52. Random scanning may be used to scan different scan region geometries with more efficiency than conventional structured scan patterns (e.g., spiral or raster patterns) due to the corresponding control and mechanical movement of the scanner associated with such structured scan patterns. For example, using a 60 MHz pulsed laser beam having pulse widths on the order of femtoseconds, a 25 μm diameter spray of scan spots may be produced on the material 50 in the region 52. Groupings of scan spots with different sizes may also be achieved.

FIG. 3 is a perspective view of a cornea 60 and a stromal bed 62 in the cornea. In one application, the pulsed laser beam may be randomly scanned at a desired region of a stromal bed 62 to create a flap in ophthalmic applications. In this embodiment, a pulsed laser beam is aimed or aligned at a predetermined location in a desired region 64 of the stromal bed 62 (e.g., along an axis, A, of the pulsed laser beam). The pulsed laser beam is then randomly scanned to produce a scanning beam (e.g., the randomized beam shown in FIG. 2), which produces a group of scan spots 66 around the predetermined location with a relatively high spot density and a random scan spot pattern. Random scanning may be used to scan the entire desired region 64 (e.g., the entire stromal bed 62) or may be used in combination with one or more scan patterns (e.g., a raster pattern, a spiral pattern, or the like) to scan the desired region 64.

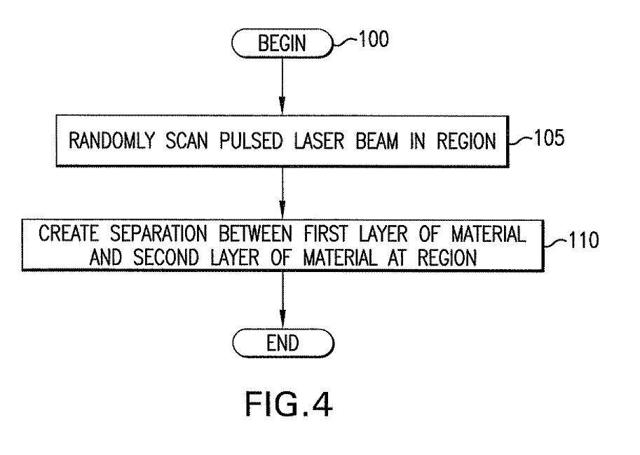

FIG. 4 is a flow diagram of a method 100 for photoaltering a desired region of a material using a pulsed laser beam in accordance with one embodiment. The pulsed laser beam is randomly scanned in the region, as indicated at 105. In one embodiment, a mirror is actuated in at least one two orthogonal directions, the pulsed laser beam is directed from the mirror to the region. Referring to FIGS. 2 and 4, for example, the mirror 40 is actuated via the piezo element 42 so as to move the mirror 40 with respect to the pivot 46. In another embodiment, a grouping of scan spots, such as the scan spots 66 shown in FIG. 3, is produced in the desired region using the pulsed laser beam. The region may be on the surface of the material or sub-surface. For example, the pulsed laser beam may be randomly scanned to produce a plurality of sub-surface scan spots in the desired region 52.

Prior to randomly scanning the pulsed laser beam, the pulsed laser beam may be aimed at a predetermined location within the region. Referring to FIGS. 3 and 4, for example, the pulsed laser beam may be aimed at a predetermined location in the desired region 64 indicated by the axis, A. In this embodiment, a spray of scan spots (e.g., the grouping of scan spots 66) is produced within the desired region 64 and localized with respect to the predetermined location. The spray of scan spots has a high scan spot density and is produced from femtoseconds pulses with low pulse energy and low average pulse energy. For example, the pulsed laser beam may be pulsed at a rate between about 30 MHz and about 1 GHz, with a pulse energy of about 800 nJ/pulse, with a pulse width of between about 300 picoseconds and about 10 femtoseconds, and/or with a wavelength between about 400 nm to about 3000 nm.

A separation is created between a first layer of the material and a second layer of the material at the region, as indicated at 110. In one embodiment, the region has a periphery, and the pulsed laser beam is scanned along the periphery prior to separating the first layer from the second layer. For example, the pulsed laser beam may be scanned as a single focal point along the periphery of the desired region to produce a sidecut (e.g., for forming a flap). In an ophthalmic application, a corneal region of the eye is randomly scanned with the pulsed laser beam, and at least a portion of the periphery of the corneal region is incised with the pulsed laser beam (e.g., to produce the sidecut).

Thus, systems 10, 41 and method 100 of photoaltering a material with a randomly scanned pulsed laser beam are disclosed. The systems 10, 41 and method 100 are suited to remove material, photoalter corneal tissue, micromachine materials, surface profile various biological tissues, or the like. Examples of some refractive eye surgery applications for the systems 10, 41 and/or method 100 include, but are not necessarily limited to, photorefractive keratectomy (PRK), LASIK, laser assisted sub-epithelium keratomileusis (LASEK), or the like. Using the randomized scanning of the systems 10, 41 and method 100 improves dissection quality and reduces the average power associated with such dissections

While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A method of photoaltering a region of a material using a pulsed laser beam, the method comprising the steps of:
randomly scanning the pulsed laser beam in the region; and
creating a separation between a first layer of the material and a second layer of the material at the region.

2. The method of claim 1, wherein the step of randomly scanning comprises:
actuating a mirror in at least one of a first direction and a second direction orthogonal to the first direction; and
directing the pulsed laser beam from the mirror to the region.

3. The method of claim 2, wherein the step of actuating a mirror comprises actuating the mirror via a piezo element.

4. The method of claim 1, wherein the step of randomly scanning comprises producing a plurality of scan spots in the region with the pulsed laser beam.

5. The method of claim 1, wherein the material has a surface, and wherein the step of randomly scanning comprises producing a plurality of sub-surface scan spots in the region with the pulsed laser beam.

6. The method of claim 1, further comprising aiming the pulsed laser beam at a predetermined location within the region prior to the step of randomly scanning, and wherein the step of randomly scanning comprises producing a spray of scan spots within the region and localized with respect to the predetermined location.

7. The method of claim 1, wherein the step of randomly scanning the pulsed laser beam comprises scanning the pulsed laser beam at a rate between about 30 MHz and about 1 GHz.

8. The method of claim 1, wherein the step of randomly scanning the pulsed laser beam comprises scanning the pulsed laser beam with a pulse energy of about 800 nJ/pulse.

9. The method of claim 1, wherein the step of randomly scanning the pulsed laser beam comprises scanning the pulsed laser beam with a pulse width of between about 300 picoseconds and about 10 femtoseconds.

10. The method of claim 1, wherein the step of randomly scanning the pulsed laser beam comprises scanning the pulsed laser beam at a wavelength between about 400 nm to about 3000 nm.

11. The method of claim 1, wherein the region has a periphery, and wherein the method further comprises, prior to the step of separating, scanning the pulsed laser beam along the periphery.

12. A system for photoaltering a region of a material, the system comprising:
a laser configured to produce a pulsed laser beam;
a controller configured to transmit a signal; and
a scanner coupled to the controller, the scanner operable to randomly scan the pulsed laser beam in the region in response to the signal.

13. The system of claim 12, wherein the scanner is further operable to direct the pulsed laser beam to a focal plane, the focal plane having a first dimension and a second dimension orthogonal to the first dimension, and wherein the scanner comprises:
a mirror configured to receive the pulsed laser beam; and
a means for randomly displacing the mirror in at least one of the first dimension and the second dimension.

14. The system of claim 12, wherein the scanner comprises:
a mirror; and
a piezo element coupled to the mirror, the piezo element configured to displace the mirror in at least one of a first direction and a second direction orthogonal to the first direction.

15. The system of claim 12, wherein the pulsed laser beam has a pulse frequency selected from a range of about 30 MHz to about 1 GHz.

16. The system of claim 12, wherein the pulsed laser beam has a pulse energy less than or equal to about 800 nanojoules/pulse.

17. The system of claim 12, wherein the pulsed laser beam has a pulse width between about 300 picoseconds and about 10 femtoseconds.

18. The system of claim 12, wherein the pulsed laser beam has a wavelength between about 400 nm to about 3000 nm.

19. The system of claim 12, wherein the scanner is further configured to selectively single-point scan the pulsed laser beam in an absence of the signal.

20. A method of forming a corneal flap of an eye, the method comprising the steps of:
randomly scanning a corneal region of the eye with a pulsed laser beam, the corneal region having a periphery; and
incising at least a portion of the periphery with the pulsed laser beam.

21. The method of claim 19, further comprising aiming the pulsed laser beam at a predetermined location within the corneal region prior to the step of randomly scanning, and wherein the step of randomly scanning comprises producing a plurality of scan spots at a predetermined depth in the corneal region with the pulsed laser beam, the plurality of scan spots being localized with respect to the predetermined location.
